(19) United States
(12) Patent Application Publication
(10) Pub. No.: US 2008/0275367 A1
(43) Pub. Date: Nov. 6, 2008

(54) Systems and methods for mapping intra-body tissue compliance

(75) Inventors: Frederico Barbagli, San Francisco, CA (US); Christopher R. Carlson, Menlo Park, CA (US)

Correspondence Address: VISTA IP LAW GROUP LLP, 12930 Saratoga Avenue, Suite D-2, Saratoga, CA, 95070, US

(73) Assignee: Hansen Medical, Inc, Mountain View, CA (US)

(21) Appl. No.: 12/150,109

(22) Filed: Apr. 23, 2008

Related U.S. Application Data
(60) Provisional application No. 60/926,020, filed on Apr. 23, 2007.

(51) Int. Cl. A61B 5/00; A61B 17/00

(52) U.S. Cl. 600/587; 606/130

(57) ABSTRACT

Robotic instrument systems and methods for generating a geometric map of an area of body tissue which is correlated with a tissue characteristic such as tissue compliance or related property. The system comprises a robotically controlled catheter which is controlled by a robotic instrument driver. A force sensor system is provided generates force signals responsive to a force applied to the distal end of the catheter. A position determination system is also provided which generates position signals responsive to the location of the distal end of the catheter. A computer is configured to receive and process the force signals and position signals to generate a geometric map of an area of body tissue correlated to the tissue compliance of different regions of the body tissue or a tissue characteristic determinable from the tissue compliance.

### RELATED APPLICATION DATA

The present application claims the benefit under 35 U.S.C. § 119 to U.S. Provisional Patent Application Ser. No. 60/926,020, filed on Apr. 23, 2007. The foregoing application is hereby incorporated by reference into the present application in its entirety.

### FIELD OF INVENTION

The invention relates generally to minimally-invasive instruments and systems, such as manually or robotically steerable catheter instrument systems, and more particularly to systems and methods for mapping and displaying intra-body tissue compliance.

### BACKGROUND

Robotic interventional systems and devices are well suited for use in performing minimally invasive medical procedures, as opposed to conventional techniques wherein the patient's body cavity is open to permit the surgeon's hands access to internal organs. Currently known minimally invasive procedures for the treatment of cardiac and other disease conditions use manually or robotically actuated instruments which may be inserted transcutaneously into body spaces such as the thorax or peritoneum, transcutaneously or percutaneously into lumens such as the blood vessels, through natural orifices and/or lumens such as the mouth and/or upper gastrointestinal tract, etc.

For example, many conventional minimally-invasive cardiac diagnostic and/or interventional techniques involve accessing the right atrium of the heart percutaneously with a catheter or catheter system by way of the inferior vena cava. When controlling an elongate instrument, such as a catheter, in any one of these applications, the physician operator can push on the proximal end of the catheter and attempt to feel the distal end make contact with pertinent tissue structures, such as the walls of the heart. Some experienced physicians attempt to determine or gauge the approximate force being applied to the distal end of a catheter due to contact with tissue structures or other objects, such as other instruments, prostheses, or the like, by interpreting the loads they tactically sense at the proximal end of the inserted catheter with their fingers and/or hands. Such an estimation of the force, however, is quite challenging and imprecise given the generally compliant nature of many minimally-invasive instruments, associated frictional loads, dynamic positioning of the instrument versus nearby tissue structures, and other factors.

The methods of detecting contact of the instrument tip with a body surface can be used in combination with localization techniques to generate a graphic, geometric map of a body structure, such as a body lumen or cavity. For example, U.S. Pat. No. 5,391,199, to Ben-Haim et al., describes that a geometric mapping of a body lumen or cavity can be performed using a manual catheter by sensing contact with a plurality of locations on the surface(s) of the lumen or cavity and using localization sensors to determine position coordinates of the instrument tip at each of the plurality of locations. This data is then used to construct a geometric map of the body lumen or cavity. U.S. Pat. No. 5,391,199, is hereby incorporated by reference herein in its entirety.

U.S. patent application Ser. No. 11/678,001, which is hereby incorporated by reference herein in its entirety, discloses robotically-navigated interventional systems including (and methods using) the capability to sense force between a distal end of a working instrument (e.g., an ablation catheter) carried in a working lumen of a robotically controlled guide instrument and the surface of an internal body cavity or lumen (referred to collectively as a “body space”). The system not only detects contact between the instrument and the surface, but also measures the magnitude of the force, also called the load. Such systems and methods can also be used to detect contact with tissue structures due to the change in the sensed force.

### SUMMARY OF THE INVENTION

Embodiments of the present invention are directed to the use of a robotically-controlled instrument system for generating a geometric mapping of an area of internal body tissue (e.g., the wall of a heart chamber), which depicts or is otherwise is correlated to tissue compliance, or a characteristic related to the tissue compliance. In various embodiments, a graphic image or model of the area of body tissue can be generated and/or displayed, with regions of the area differentiated based upon the measured tissue compliance or a characteristic of the tissue that is determined based upon the measured tissue compliance. By way of non-limiting example, the tissue compliance may be used to determine tissue type, such as bone, soft tissue, myocardial wall, etc. In one embodiment, a graphically rendered image of the map depicts a geometric map of the tissue area (e.g., a chamber of the heart), with corresponding respective tissue types displayed in a different color, shade, or other demarcation as determined from their respective compliance.

In one embodiment, a robotically-controlled instrument system includes an elongate flexible guide instrument coupled to an instrument driver. The guide instrument defines a working lumen or channel through which an electrophysiology (e.g., mapping and/or ablation) catheter may be positioned through a proximal end opening of the guide instrument in communication with the working lumen. The catheter is inserted through the length of the guide instrument lumen, until a distal end of the catheter extends out of a distal opening of the guide instrument in communication with the lumen. The guide instrument is inserted into a patient's body (the catheter may be inserted into the guide instrument before or after it has been inserted into the body), with a bendable distal end portion of the guide instrument positioned in a selected anatomical workspace to be mapped (or for which a wall portion or other tissue structure is to be mapped). The distal end portion of the guide instrument is maneuvered within the workspace, with the distal end of the catheter periodically contacting a tissue structure or surface within or bordering the workspace. A force sensor or sensing apparatus is coupled to a proximal end of the catheter (i.e., proximal of the guide instrument), and senses a force (or “load”) met by the catheter when it comes into contact with the tissue wall or structure. In alternate embodiments, the force sensor may take on numerous different configurations and can be positioned at various locations along the catheter (e.g., built into the tip, or a strain gage provided in a wall of the catheter), such as a load sensor, pressure sensor or other suitable sensor located at or near the distal end of the guide catheter. The force sensor generates force signals responsive to the force applied to the distal end of the guide catheter when it contacts a tissue surface.

The instrument system further includes or is otherwise operatively coupled with a localization (or “position determining”) system for determining the relative position of the distal end of the catheter as it contacts a tissue surface or structure. The position determining system generates position signals which are responsive to the position of the catheter as it is moved to a plurality of locations on an area of body tissue. The position determining system may be any suitable system, including without limitation, localization systems such as those which use magnetic sensors and antenna, open loop or closed loop position systems, shape sensing system such as Bragg fiber optic systems, etc.

The position determining system and force sensor are operatively coupled to a suitable processor (e.g., a system controller or associated computer), a well as associated signal conditioning electronics (collectively, “computer assembly”), with is preferably coupled to a graphic display. The computer assembly is configured to receive and process the position data to generate a geometric map of a tissue surface or other structure based on the localization data provided by the position determining system. The computer assembly is also configured to receive and process the force signals and to calculate a relative compliance of the tissue being contacted by the distal end of the catheter at each of the contact locations. The computer assembly can then generate and display a geometric map correlated with the tissue compliance of the tissue at various regions of the area of tissue of interest.

The method of mapping an area of body tissue with the robotic instrument system is fairly straightforward. The guide instrument is introduced into a patient's body. Then, the distal end of the guide catheter is robotically maneuvered into contact with a plurality of locations on an area of body tissue at an interventional procedure site. The robotic instrument system may maneuver the distal end to the plurality of locations in an automated manner (e.g., moving around the heart chamber or other anatomical space and automatically collecting position data and tissue compliance data needed to render a map). Alternatively, a physician may drive the catheter by giving commands to the robotic instrument system to go to a particular location, and then to move the distal end of the guide catheter into contact with a plurality of locations on the body tissue. This may be with an organ such as the patient's heart or kidney, or other body lumen such as an artery, or any other body structure. As the distal end of the catheter is moved in to contact with each location on the body tissue, the force on the tip and the deflection of the tissue due to the force is sensed by the system in order to determine the tissue compliance of the tissue at each location. At substantially the same time, the position of each of the locations on the body tissue is also determined. This data is then used to generate a geometric map of the body tissue which is representative of the tissue compliance of the tissue. Further, the tissue compliance may be used to determine other tissue characteristics such as the type of tissue, condition of the tissue, or other characteristic. For example, one region of the tissue may be very elastic or squishy which may be indicative of soft tissue, while another region may be more firm, indicative of muscle tissue or bone. Then, the generated map can show a graphic image of the area of body tissue with the regions of different tissue characteristics demarcated, such as being shown in different shades, colors, cross-hatching, labels or other suitable graphic indication.

The map may then be used in planning and performing a surgical procedure (including diagnosis and treatment procedures), with the same robotic instrument system or other surgical instruments.

### BRIEF DESCRIPTION OF THE DRAWINGS

The drawings illustrate the design and utility of illustrated embodiments of the invention, in which similar elements are referred to by common reference numerals.

FIG. 1 illustrates one embodiment of a robotic instrument system according to the present invention;

FIG. 2 illustrated one embodiment of a catheter assembly used in the robotic instrument system of FIG. 1;

FIG. 3 illustrates a schematic representation of a robotic catheter having a force sensor system which utilize dithering;

FIG. 4 illustrates one embodiment of a guide catheter having a force sensor system according to the present invention; and

FIG. 5 illustrates another embodiment of a guide catheter having a force sensor system according to the present invention.

### DETAILED DESCRIPTION OF THE ILLUSTRATED EMBODIMENTS

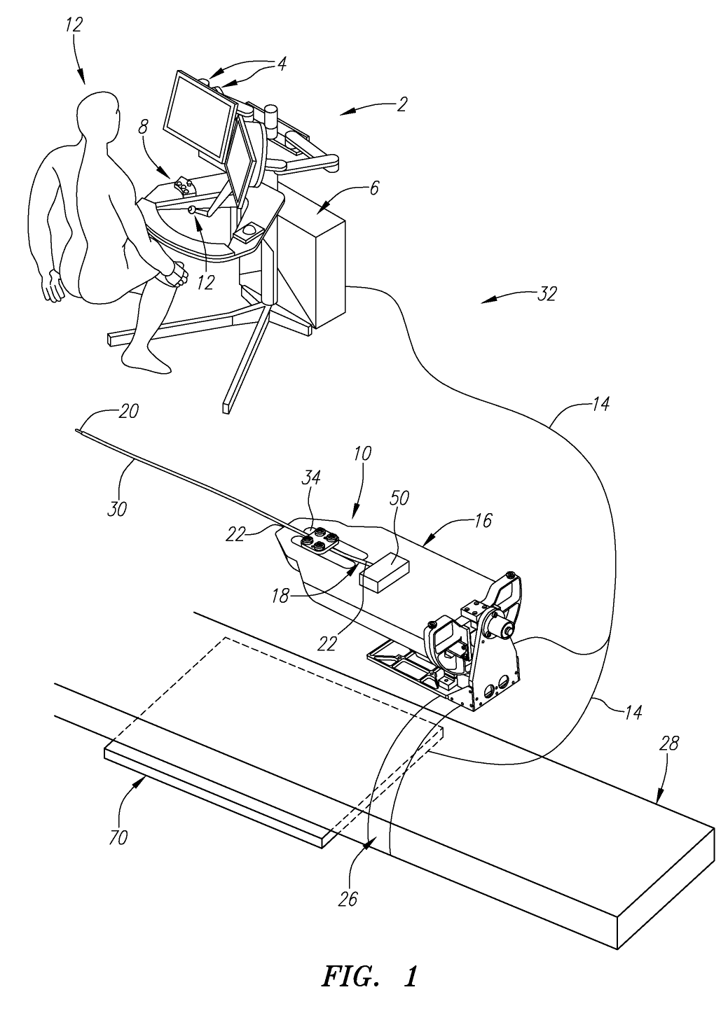

Embodiments of the present invention are directed to robotic instrument systems and methods of their use for creating a geometric map of an area of body tissue which displays an image of the area of body tissue with tissue compliance or related characteristics at various regions of the map superimposed thereon. One illustrative embodiment a robotic instrument system (32) according to the present invention is shown in FIG. 1. In addition to the above-incorporated U.S. patent application Ser. No. 11/678,001, exemplary embodiments of robotic instrument systems that may be modified for constructing and using embodiments of the present invention are disclosed and described in detail in the following U.S. patent applications, which are all incorporated herein by reference in their entirety: U.S. patent application Ser. No. 11/073,363, filed Mar. 4, 2005; U.S. patent application Ser. No. 11/179,007, filed Jul. 6, 2005; U.S. patent application Ser. No. 11/418,398, filed May 3, 2006; U.S. patent application Ser. No. 11/481,433, filed Jul. 3, 2006; U.S. patent application Ser. No. 11/637,951, filed Dec. 11, 2006; and U.S. patent application Ser. No. 11/640,099, entitled “Robotic Catheter System and Methods”, filed Dec. 14, 2006; and U.S. Provisional Patent Applications Nos. 60/833,624 (filed Jul. 26, 2006) and 60/835,592 (filed Aug. 3, 2006), both entitled, “Robotic Catheter System and Methods.”

The robotic instrument system (32) includes an operator control station (2) located remotely from an operating table (22), and a robotic catheter assembly (10).

The control station (2) comprises a user interface (8) that is operatively connected to the robotic catheter assembly (10). A physician or other user (12) interacts with the user interface (8) to operate the robotic catheter assembly (10). The user interface (8) is connected to the robotic catheter assembly (10) via a cable (14) or the like, thereby providing one or more communication links capable of transferring signals between the operator control station (2) and the robotic catheter assembly (10). Alternatively, the user interface (8) may be located in a geographically remote location and communication is accomplished, at least in part, over a wide area network such as the Internet. Of course the user interface (8) may also be connected to the robotic catheter assembly (10) via a local area network or even wireless network that is not located at a geographically remote location.

The control station (2) also comprises a display (4) that is used to display various aspects of the robotic instrument system (2). For example, an image of the working instrument and guide instrument (described in further detail below) may be displayed in real time on the display (4) to provide the physician (12) with the current orientation of the various devices as they are positioned, for example, within a body lumen or region of interest. The control station (2) further comprises a computer assembly (6), which may comprise a personal computer or other type of computer work station for performing the data processing operations disclosed herein.

The robotic catheter assembly (10) is coupled to the operating table (22) by an instrument driver mounting brace (26). The robotic catheter assembly (10) comprises a robotic instrument driver (16), a working catheter (18), and a guide catheter (30) (also referred to herein as an instrument guide catheter, guide catheter, robotic guide instrument, robotic guide catheter, or the like). The instrument driver mounting brace (26) of the depicted embodiment is a relatively simple, arcuate-shaped structural member configured to position the instrument driver (16) above a patient (not shown) lying on the table (22).

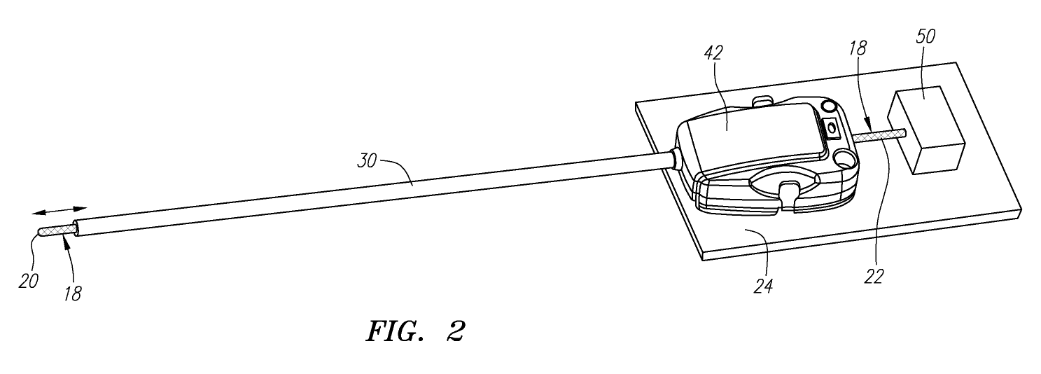

Referring further to FIG. 2, the catheter (18) is typically an elongate, flexible device configured to be inserted into a patient's body. The catheter (18) has a distal end (20) and a proximal end (22). As non-limiting examples, an catheter (18) may comprise an intravascular catheter, an endoscopic surgical instrument or other medical instrument. The catheter (18) is configured to be operable via the instrument driver (16) such that the instrument driver (16) can operate to steer the catheter (18) and also to operate tools and devices (also called end effectors) which may be provided on the instrument assembly (18) (e.g. an imaging device or cutting tool disposed on the distal end of the catheter (18). The working catheter (18) may be movably positioned within the working lumen of the guide catheter (30) to enable relative insertion of the two instruments, relative rotation, or “roll” of the two instruments and relative steering or bending of the two instruments relative to each other, particularly when the distal end (20) of the working catheter (18) is inserted beyond the distal tip of the guide catheter (30). The system (32) also comprises a mechanical ditherer (50) or other dithering mechanism or device as described herein.

The guide catheter (30) is mounted via a base (24) carrying the ditherer (50). The ditherer (50) is coupled to the working catheter (18) that is dithered back-and-forth relative to the guide catheter (30). The guide catheter (30) is coupled to a housing (42) that mechanically and electrically couples the guide catheter (30) to a robotically-controlled manipulator. For example, the guide catheter (30) may be coupled to a robotically controlled instrument driver such as, for instance, the DA VINCI surgical system sold by Intuitive Surgical, Inc. of Sunnyvale, Calif.

Referring back to FIG. 1, the system (32) also comprises a force sensor system (34) for sensing the force on the distal end (20) of the working catheter (18). The force sensor system (34) may be disposed at various locations, including a force sensor placed on the distal end (20) of the working catheter (18), a force sensor placed on the proximal end (22) of the working catheter (18), a ditherer force sensor system (as described in more detail below) located at the proximal end of the working catheter (18), or other suitable location. The force sensor system (34) is in operable communication with the operator control station (2) via the communication link (14).

The system (32) further comprises a position determining system (70) for determining the position of the distal end (20) of the working catheter (18). The position determining system (70) may be any suitable localization system, such as the electromagnetic localization sensing systems available from Biosense, Inc., Ascension Technologies, or St. Jude Medical, which are capable of sensing the locations of a plurality of sensors (72) located on the catheter (18). The position determining system (70) is in operable communication with a computer assembly (6) of the operator control station (2) through the communication link (14). The computer assembly (6) may comprise conditioning electronics for conditioning the force signals from the force sensor system (34) and the position signals from the position determining system (70).

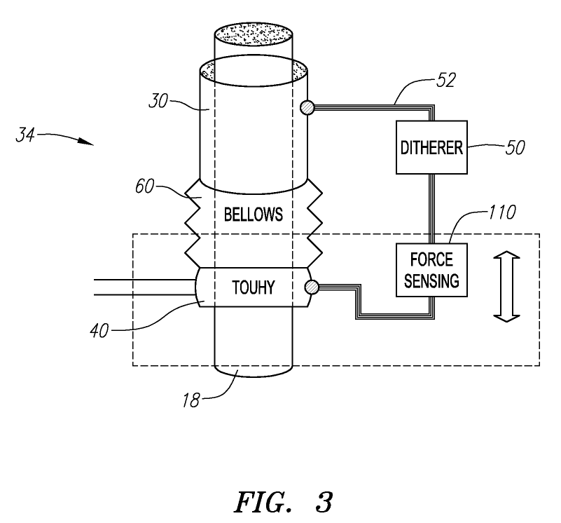

Turning now to FIG. 3, one embodiment of a force sensor system (34) intended to be located at the proximal end (22) of the catheter (18) will be described. FIG. 3 is a schematic illustration of a system and method for measuring a force on the distal end (20) of the catheter (18) using a dithering technique. In this embodiment, the working catheter (18) dithers with respect to substantially stationary guide catheter (30). In order to dither the working catheter (18) back and forth (longitudinally), the mechanical ditherer (50) will drive the working catheter (18) through a force sensor (110), which will measure the direct force needed to insert and withdraw the working catheter (18) in and out of the guide catheter (30). The ditherer (50) is mechanically grounded (via a mechanical linkage 52) to a proximal region of the guide catheter (30) and is thus stationary relative to the guide catheter (30), but the force sensor (10) and working catheter (18) move together relative to the guide catheter (30). The force signals from the force sensor (110) are transmitted to the computer assembly (6) for data processing. This type of force sensor system (34) is described in U.S. patent application Ser. No. 11/678,001, along with various other embodiments of dithering force sensors. The disclosure of U.S. patent application Ser. No. 11/678,001 is hereby incorporated by reference herein in its entirety.

In the embodiment illustrated in FIG. 3, the ditherer (50) and force sensor (110) are mechanically linked to a seal (40), such as a Touhy seal. The Touhy seal (40) acts as a fluidic seal which can add significant and erratic drag to the reciprocating in-and-out motion of the working catheter (18), which would adversely affect the accuracy of readings from the force sensor (110). This embodiment eliminates this effect by mechanically securing or locking the Touhy seal (40) to the working catheter (18) so the two are dithered together. In addition, FIG. 3 illustrates the flexible bellows (60) that is connected to the proximal end of the guide catheter (30) at one end and secured to the Touhy seal (40) at the other end. The bellows (60) expands and contracts like an accordion with the dithering motion. The bellows (60) advantageously applies a very low drag force on the working catheter (18) during the dithering motion as opposed to the high drag force that would be applied if the working catheter (18) was dithered through the Touhy seal (40).

By “dithering” the working catheter (18) with respect to the guide catheter (30), the repeated cyclic motion may be utilized to overcome frictional challenges normally complicating the measurement, from a proximal location, of loads at the distal end (20) of the working catheter (18) when in contact with a surface. In one embodiment, the dithering motion may be applied on a proximal region of the working catheter (18) as is illustrated in FIG. 1. In other words, for example, if a user were to position a working catheter (18) down a lumen of a guide catheter (30) so that the distal end (20) of the working catheter (18) is sticking out slightly beyond the distal end of the guide catheter (30) (as shown in FIG. 2), and have both the guide catheter (30) and working catheter (18) advanced through the blood vessel(s) from a femoral location to the chambers of the heart, it may be difficult to sense contact(s) and force(s) applied to the distal end (20) of the working catheter (18) due to the complications of the physical relationship with the associated guide catheter (30). In particular, in a steady state wherein there is little or no relative axial or rotational motion between the working catheter (18) and guide catheter (30), the static coefficient of friction is applicable, and there are relatively large frictional forces keeping the working catheter (18) in place relative to the guide catheter (30) (no relative movement between the two).

To release this relatively tight coupling and facilitate proximal measurement of forces applied to the distal end (22) of the working catheter (18), dithering motion may be used to effectively break loose this frictional coupling. In one embodiment, such as the one illustrated in FIG. 3, the dithering motion may be applied on a proximal region of the working catheter (18). In still other embodiments (not shown), it may be possible to dither the guide catheter (30) with respect to a stationary or substantially stationary working catheter (18). In yet another embodiment, both the working catheter (18) and guide catheter (30) may be dithered with respect to one another.

While the embodiment illustrated in FIG. 3 shows the operation of the ditherer 50 with respect to a catheter assembly that includes both a working catheter and a guide catheter, it should be appreciated that the functionality of the working and guide catheters can be incorporated into a single catheter to which the ditherer 50 is operatively coupled.

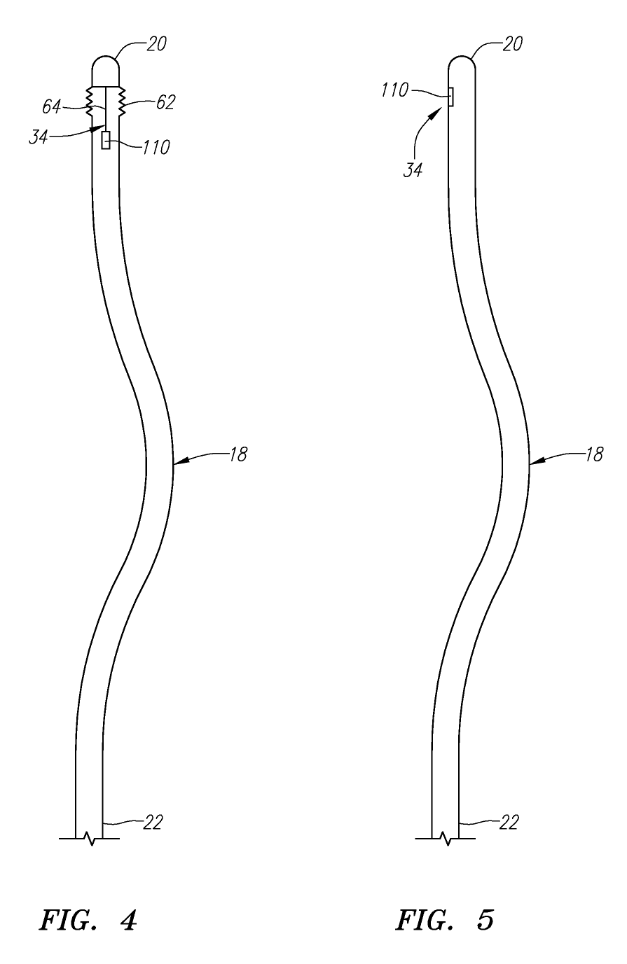

The issues presented by the frictional forces and other complexities associated with a force sensor located at the proximal end (22) of the working catheter (18) may be eliminated by locating the force sensor at or near the distal end (20) of the working catheter (18). FIGS. 4 and 5 illustrate two exemplary embodiments. Referring first to the embodiment of FIG. 4, a force sensor system (34) is located at or near the distal end (20) of the working catheter (18). The force sensor system (34) comprises a flexible bellows (62) that expand and contract in response to a force placed on the distal end (22). A transfer rod (64) is coupled at one end to the distal end (22) and at the other end to a force sensor (110). The force sensor (110) may be any suitable force sensor such as a load sensor, pressure sensor, piezoelectric sensor, strain gauge or the like. When the distal end (22) contacts body tissue, the bellows is compressed causing the transfer rod (64) to push on the force sensor (110). The force sensor (110) transmits a force signal responsive to the amount of force being applied to the distal end (22) to the computer assembly (6).

The working catheter (18) of FIG. 5 is similar to that of FIG. 4, except that the force sensor system (34) is applied directly onto the shaft or even the distal end (20) of the working catheter (18). The force sensor system (34) comprises a force sensor (110), which transmits a force signal to the computer assembly (6). In this embodiment, the strain of the working catheter (18) itself is used to measure the force being applied to the distal end (18). Although each of the different types of force sensors (110) described herein may be utilized, a strain gauge may be the most suitable for this embodiment.

As briefly discussed above, the computer assembly (6) is configured to receive and process the force signals from the force sensor system (34) and the position signals from the position determining system (70). It should be understood that the computer assembly (6) may comprise one or more computers, signal conditioning electronics, and other displays and peripherals. The computer assembly (6) is also configured to process the force signals and position signals to generate a geometric map of an area of body tissue correlated to the tissue compliance of the tissue or other characteristic of the body tissue related to its tissue compliance. As an example, as the working catheter (18) is robotically maneuvered within a patient's body at an area of interest, the distal end (20) is moved into contact with the plurality of locations on the area of body tissue. The computer assembly (6) receives the position signals and force signals and determines the force on the tip, the deflection of the tissue and the position of the location on the area of body tissue at each of the plurality of locations. The computer assembly (6) is further configured to generate a geometric map of the area of body tissue using the position determined for each location, and to also correlate the tissue compliance at each location and superimpose the tissue compliance on the geometric map. Regions of different compliance may be superimposed on the mapping in different colors, shades or other suitable representation. The computer assembly (6) may also be configured to relate the measured compliance of the different regions of the area of tissue to other tissue characteristics, such as tissue type, tissue condition (necrosed, healthy, diseased, etc.) or other characteristic of interest.

The robotic instrument system (32) may maneuver the distal end to the plurality of locations in an automated manner in response to a programmed path or target (e.g., moving around the heart chamber or other anatomical space and automatically collecting position data and tissue compliance data needed to render a map). Alternatively, a physician may drive the working catheter (18) by giving commands to the robotic instrument system (32) to move the working catheter (18) to a particular location, and then to move the distal end (20) of the working catheter (18) into contact with a plurality of locations.

As an example, the human heart is composed of three primary types of tissue: the myocardium which is the muscular tissue of the heart; the endocardium which is the inner lining of the heart; and the epicardium which is a connective tissue layer around the heart. These tissues have inherently differing elastic properties, e.g. the myocardium tissue is firmer than the endocardium tissue. Accordingly, to map a patient's heart, the working catheter (18) can be advanced into the heart of a patient, the distal end (20) is then contacted with a plurality of locations within the heart, and a map can be generated showing an image of the different structures of the heart.

While multiple embodiments and variations of the many aspects of the invention have been disclosed and described herein, such disclosure is provided for purposes of illustration only. Many combinations and permutations of the disclosed system are useful in minimally invasive surgery, and the system is configured to be flexible. Thus, it should be understood that the invention generally, as well as the specific embodiments described herein, are not limited to the particular forms or methods disclosed, but also cover all modifications, equivalents and alternatives falling within the scope of the appended claims.

What is claimed is:

1. A robotically controlled instrument system, comprising:
an elongate instrument having a distal end surface adapted to make contact with an internal body tissue surface;
a force sensor associated with the instrument, wherein the force sensor generates force signals responsive to a force applied to the distal end surface of the instrument upon contacting a tissue surface, the force signals corresponding to a respective compliance of tissue being contacted by the instrument distal end surface;
a position determining system which generates position data indicative of a position of the distal end of the instrument;
a processor operatively coupled to the force sensor and position determining system, and configured to process the respective force signals and position signals to generate a geometric map of an area of body tissue correlated with a tissue characteristic related to tissue compliance.

2. The system of claim 1, wherein the tissue characteristic is tissue compliance.

3. The system of claim 1, wherein the instrument comprises a robotically controlled guide instrument.

4. The system of claim 3, wherein the force sensor is coupled to a distal end portion of the guide instrument.

5. The system of claim 1, wherein the instrument comprises a catheter carried in a robotically controlled guide instrument.

6. They system of claim 5, wherein the force sensor is coupled at a proximal end portion of the catheter.

7. The system of claim 1, further comprising a display coupled to the processor, wherein the processor causes regions of the body tissue area in the map having differences in tissue compliance to be visually highlighted on the display.

8. The system of claim 7, wherein the body tissue regions having differences in tissue compliance to be visually highlighted using different colors or shades of colors on the map.

9. The system of claim 1, wherein the position determining system comprises at least one position sensor disposed on the instrument.

10. A method of mapping a relative tissue compliance of an area of body tissue, comprising:
robotically maneuvering an elongate instrument within an internal body space;
advancing a distal end surface of the instrument into contact with each of a plurality of locations on the area of body tissue;
sensing the compliance of the tissue at each of the plurality of locations on a tissue surface;
determining a position of each of the plurality of tissue surface locations;
generating a geometric map of the area of tissue representative of a characteristic of the tissue related to a compliance of the tissue.

11. The method of claim 10, wherein the tissue characteristic is tissue compliance.

12. The method of claim 10, wherein tissue compliance is measured using a force sensor coupled to the instrument.

13. The method of claim 12, wherein the force sensor is coupled to a proximal portion of the instrument.

14. The method of claim 12, wherein the force sensor is coupled to a distal portion of the instrument.
